(19) United States
(12) Patent Application Publication
(10) Pub. No.: US 2009/0198341 A1
(43) Pub. Date: Aug. 6, 2009

(54) BONE PROSTHESIS

(75) Inventors: George Y. CHOI, Redwood City, CA (US); James Brian WARNE, Los Gatos, CA (US)

Correspondence Address: LEVINE BAGADE HAN LLP, 2400 GENG ROAD, SUITE 120, PALO ALTO, CA, 94303, US

(73) Assignee: Talus Medical, Inc., Redwood City, CA (US)

(21) Appl. No.: 12/203,015

(22) Filed: Sep. 2, 2008

Related U.S. Application Data
(63) Continuation of application No. PCT/US2007/063233, filed on Mar. 2, 2007.
(60) Provisional application No. 60/779,307, filed on Mar. 2, 2006.

(51) Int. Cl. A61F 2/42

(52) U.S. Cl. 623/21.18

(57) ABSTRACT

A partial bone prosthesis and methods of using the same are disclosed herein. The partial bone prosthesis can be configured for use, for example, on the superior side of the talus or an end of a long bone, such as the tibia. The partial bone prosthesis can anchor to the perimeter of the contact patch between the bone and the prosthesis. For example, the prosthesis can be attached to the talus without drilling holes through the talus, preserving structural integrity and blood flow within the talus.

### CROSS-REFERENCE TO RELATED APPLICATIONS

This application is a continuation of PCT International Application No. PCT/US2007/063233 filed Mar. 2, 2007 which claimed benefit of priority to U.S. Provisional Application No. 60/779,307 filed Mar. 2, 2006, both of which are incorporated by reference herewith in their entirety.

### FIELD OF THE INVENTION

This invention relates to a bone prosthesis and methods of using the same. More specifically, the bone prosthesis can be anchored perimeterly.

### BACKGROUND OF THE INVENTION

Osteoarthritis or trauma can result in ankle pathology of uneven wear on, and/or direct trauma to, the surface of the talus. This commonly leads to cartilage erosion and subsequent break down of subchondral bone. Osteoarthritis and certain trauma on the talus are often treated by fusing the talus to the tibia. This fusion procedure results in loss of mobility of the ankle, and the expected complications resulting from a loss of mobility including gait changes, further stress-related injuries, and a reduction of the patient's overall mobility.

A secondary treatment for osteoarthritis in the talus—and in other bones—is to replace part of the damaged bone with a partial bone prosthesis. The partial bone prostheses, such as those for the talus or the long bones (e.g., femur, tibia, humerus, ulna), typically anchor into the remainder of the bone. This anchoring occurs through a radial center with respect to the longitudinal axis of the long bone, or roughly with respect to the vertical in the case of the talus.

FIGS. 1 and 2 illustrate anterior and lateral views of the tibia 6, talus 12 and fibula 2 (not shown in FIG. 2). A vertical axis 8 and an original talus thickness 10 is shown. The original talus thickness 10 is dependent on individual anatomical factors and the amount of pathological bone degradation.

FIGS. 3 and 4 illustrate that when the talus 12 is prepared for a typical prosthesis 20 implantation, tissue, such as a superior surface and adjacent bone, of the talus 12 is removed. The remaining portion of the talus 12 has a new superior surface (i.e., contact patch 16 for the prosthesis 20) substantially perpendicular to the vertical axis 8. The minimum talus height after tissue removal (i.e., minimum cut-down thickness 14) is substantially less than the minimum tissue height of the original talus 12, as shown in FIGS. 1 and 2.

FIGS. 5 and 6 illustrate that the typical partial bone prosthesis is attached to the contact patch 16. The prostheses 20 often have central anchors 22 that protrude into and/or through the center of the talus 12 along or near the vertical axis 8 With the prosthesis 20 implanted, the post-implant talus minimum thickness 18 is very small and increases the fragility of the talus 12 during and after the implantation of the prosthesis 20. The damage done by the central anchors 22 can also impair blood flow to and within the talus 12.

Therefore, a need exists for a partial bone prosthesis that does not substantially impact the minimum bone thickness or minimum longitudinal or vertical thickness after implanting in a bone. There also exists a need for a partial bone prosthesis that does not anchor centrally to the prosthesis.

### SUMMARY OF THE INVENTION

A partial bone prosthesis is disclosed herein. The partial bone prosthesis can be used to treat a long bone, talus, knee, hip, shoulder, elbow, or vertebra.

The prosthesis can have a prosthesis body having a central axis. The prosthesis body can have a perimeter anchor. The perimeter anchor can be radially distal to the central axis. The perimeter anchor can be substantially thicker than the remainder of the prosthesis body.

The perimeter anchor can form a complete perimeter around the central axis or the perimeter anchor can forms an incomplete perimeter around the central axis. The prosthesis body can have a branch. The branch can have all or part of the perimeter anchor.

The perimeter anchor can have an ingrowth matrix. The perimeter anchor can have a first ridge. The perimeter anchor can have a second ridge. The perimeter anchor can have a supplemental anchor port. The prosthesis body can be made from a titanium alloy, a cobalt chromium alloy, or combinations thereof.

A method for implanting a partial bone prosthesis to a bone end of a bone having a central axis is also disclosed. The method includes removing tissue from the bone end. Removing tissue includes removing substantially thicker tissue distally front the central axis than proximally to the central axis. Removing tissue also includes exposing a contact patch on the bone. The method includes deploying the partial bone prosthesis to the contact patch.

Deploying can include applying a bone morphogenic protein to the bone and/or the prosthesis. Deploying can include inserting a supplemental anchor through the partial bone prosthesis and into the bone. Inserting can include inserting the supplemental anchor through the perimeter anchor.

### BRIEF DESCRIPTION OF THE DRAWINGS

FIG. 1 is not the invention and illustrates an anterior view of the bones of the upper ankle.

FIG. 2 is not the invention and illustrates a lateral view of FIG. 1 sans fibula.

FIG. 3 is not the invention and illustrates the bones of FIG. 1 after typical tissue removal in preparation for implanting a partial talus prosthesis.

FIG. 4 is not the invention and illustrates the bones of FIG. 2 after typical tissue removal in preparation for implanting a partial talus prosthesis.

FIG. 5 is not the invention and illustrates FIG. 3 after implanting a known partial talus prosthesis.

FIG. 6 is not the invention and illustrates FIG. 4 alter implanting a known partial talus prosthesis.

FIGS. 7 through 9 are perspective views of variations of the talus bone prosthesis.

FIGS. 10a and 11a illustrate cross-sections A-A and B-B, respectively, of a variation of the bone prosthesis.

FIGS. 10b and 11b illustrate cross-sections A-A and B-B, respectively, of a variation of the partial bone prosthesis.

FIGS. 12 and 13 illustrate cross-sections A-A and B-B, respectively, of a variation of the partial bone prosthesis.

FIGS. 14 and 15 illustrate cross-sections A-A and B-B, respectively, of a variation of the partial bone prosthesis.

FIGS. 16 and 17 illustrate cross-sections A-A and B-B, respectively, of a variation of the partial bone prosthesis.

FIGS. 18 and 19 illustrate cross-sections A-A and B-B, respectively, of a variation of the partial bone prosthesis.

FIGS. 20 and 21 illustrate cross-sections A-A and B-B, respectively, of a variation of the partial bone prosthesis.

FIG. 22a is a front view of the variation of the bone prosthesis of FIG. 9.

FIG. 22b is a bottom view of the variation of the bone prosthesis of FIG. 9.

FIG. 22c is a side view of the variation of the bone prosthesis of FIG. 9.

FIG. 23 illustrates a variation of a prosthesis floating component.

FIG. 24a is a top view of the prosthesis floating component of FIG. 23.

FIG. 24b is a side view of the prosthesis floating component of FIG. 23.

FIG. 24c illustrates a variation of section C-C of FIG. 24b.

FIG. 25 illustrates a variation of the prosthesis tibia component.

FIG. 26a is a side view of the prosthesis tibia component of FIG. 25.

FIG. 26b is a front view of the prosthesis tibia component of FIG. 25.

FIG. 27 is an anterior view of a variation of a method for removing tissue for preparing the bones of FIG. 1 for implanting a variation of the partial bone prosthesis.

FIG. 28 is a lateral view of the variation of the method shown in FIG. 27 for removing tissue for preparing the bones of FIG. 2 for implanting a variation of the partial bone prosthesis.

FIG. 29 is an anterior view of a variation of a method for implanting the partial bone prosthesis.

FIG. 30 is a lateral view of the variation of the method shown in FIG. 29 for implanting the partial bone prosthesis.

FIG. 31 is an anterior view of a variation of a method for implanting the partial bone prosthesis with ridges.

FIG. 32 is a lateral view of the variation of the method shown in FIG. 31 for implanting the partial bone prosthesis.

FIG. 33 is an anterior view of a variation of a method for implanting the partial bone prosthesis with supplemental anchors.

FIG. 34 is a lateral view of the variation of the method shown in FIG. 33 for implanting the partial bone prosthesis.

FIGS. 35 and 36 are anterior views of various variations of methods for implanting a multi-component variation of the partial bone prosthesis.

FIG. 37 is a lateral view of the variations of the method shown in FIGS. 35 and 36 for implanting the partial bone prosthesis.

FIGS. 38 and 39 are anterior views of various variations of methods for implanting a multi-component variation of the partial bone prosthesis.

FIG. 40 is a lateral view of the variations of the method shown in FIGS. 38 and 39 for implanting the partial hone prosthesis.

FIG. 41 illustrates a variation of a method of implanting a multi-component variation of the partial bone prosthesis in a knee.

FIG. 42 is an exploded view of a variation of a method of implanting a multi-component variation of the partial bone prosthesis in a hip.

FIG. 43 illustrates a variation of a method of implanting a multi-component variation of the partial bone prosthesis in a shoulder.

### DETAILED DESCRIPTION

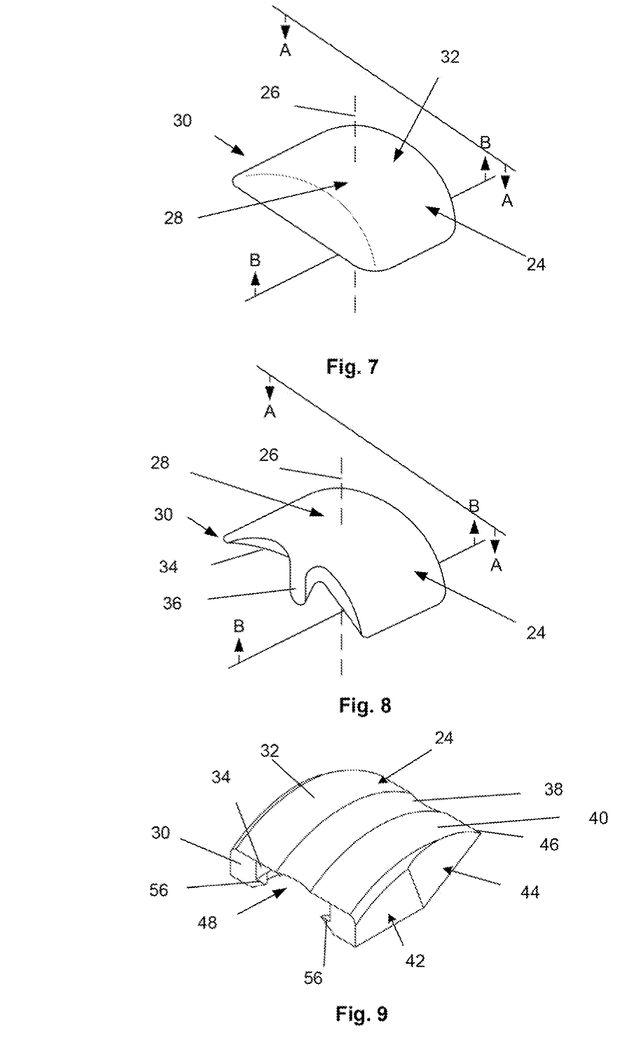

FIG. 7 illustrates that the partial bone prosthesis can have a prosthesis body 24. The prosthesis body 24 can have a central axis 26. During use in a long bone, the central axis 26 can be substantially parallel and/or aligned with a longitudinal axis of the long bone. During use in the talus 12 or in a vertebra, the central axis 26 can be substantially parallel and/or aligned with a vertical axis 8.

The prosthesis body 24 can have a central portion 28. The central axis 26 can pass through the central portion 28. The prosthesis body 24 can have a perimeter anchor 30. The perimeter anchor 30 can be radially distal to the central axis 26. The perimeter anchor 30 can partially or completely surround the central portion 28.

The prosthesis can have a distal prosthesis surface 32. The distal prosthesis surface 32 can be configured to substantially match the exterior of the portion of the bone being replaced by the prosthesis. The proximal and distal prosthesis surfaces 32 are proximal and distal, respectively, to the remainder of the bone which is being partially replaced.

FIG. 8 illustrates that the prosthesis body 24 can have one or more branches 36. The branches 36 can extend radially from the central axis 26. The branches 36 can extend substantially parallel, or not substantially parallel, to the central axis 26 at a radius from the central axis 26.

The prosthesis can have a proximal prosthesis surface 34. The proximal prosthesis surface 34 can be configured to attach to the bone.

FIG. 9 illustrates that the prosthesis body 24 can have one or more grooves 38 extending along a fore-aft (i.e., front-back or anterior-posterior) axis on the distal prosthesis surface 32. The groove 38 can be laterally centered on the prosthesis body 24. The groove 38 can be configured to align with a tongue in an adjacent implant or a protrusion in an adjacent bone to the groove. The groove 38 can be configured to minimize or otherwise restrict lateral movement of the implant with respect to the adjacent implant or adjacent hone to the groove.

The distal prosthesis surface 32 can have one or more shoulders 40 on each side of the groove 38 and between grooves 38. The shoulders 40 can be flat and/or curved surfaces. The shoulders 40 and/or the grooves 38 can have low-friction coating, for example made from PTFE (e.g., Teflon & from E.I. du Pont de Nemours and Company of Wilmington, Del.).

The prosthesis body 24 can have a prosthesis flat 42 and a prosthesis rise 44. The prosthesis rise 44 can extend at an angle from the prosthesis flat 42 with measured parallel the up-down (i.e., dorsal-plantar or dorsal-palmar) axis.

The prosthesis body 24 can have a sharp edge 46 at the front and/or back of the prosthesis body 24. The prosthesis body 24 can have a flat, blunt face at the front and/or back of the prosthesis body 24.

The prosthesis body 24 can have a body channel 48. The bone channel 48 can pass through the prosthesis body 24 from the front to the back or from a first lateral side (i.e., left) to a second lateral side (i.e., right). The surface of the bone channel 48 can be formed by the proximal prosthesis surface 34. The perimeter anchor 30 can extend along two opposite sides of the bone channel 48. The perimeter anchor 30 can be vacant at the front port and/or back port of the bone channel 48.

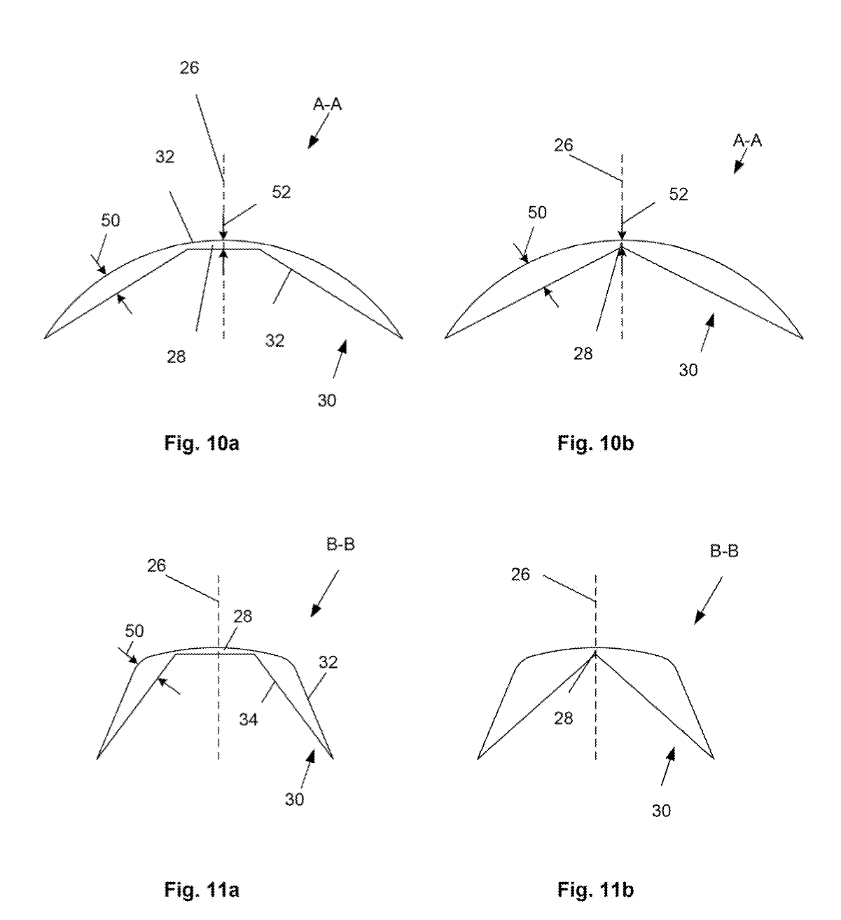

FIGS. 10a and 11a illustrates that the perimeter anchor 30 can have a maximum anchor thickness 50. The central portion 28 (i.e., not the perimeter anchor 30) can have a maximum central portion thickness 52. The maximum anchor thickness 50 can be substantially larger than the maximum central portion thickness 52.

The proximal prosthesis surface 34 at the central portion 28 can form an angle with the proximal prosthesis surface 34 at the perimeter anchor 30. The distal prosthesis surface 32 can be substantially or entirely curved. The central portion 28 can have a substantial radius from the central axis 26.

The perimeter anchor 30 can have a maximum anchor thickness 50. The central portion 28 can have a maximum central portion thickness 52. The maximum anchor thickness 50 can be substantially greater than the maximum central portion thickness 52.

FIGS. 10b and 11b illustrate that the perimeter anchor 30 can substantially converge at the central axis 26. The proximal prosthesis surface 34 can peak and/or form an angle at the central axis 26. The central portion 28 can have a nominal or insubstantial radius from the central axis 26.

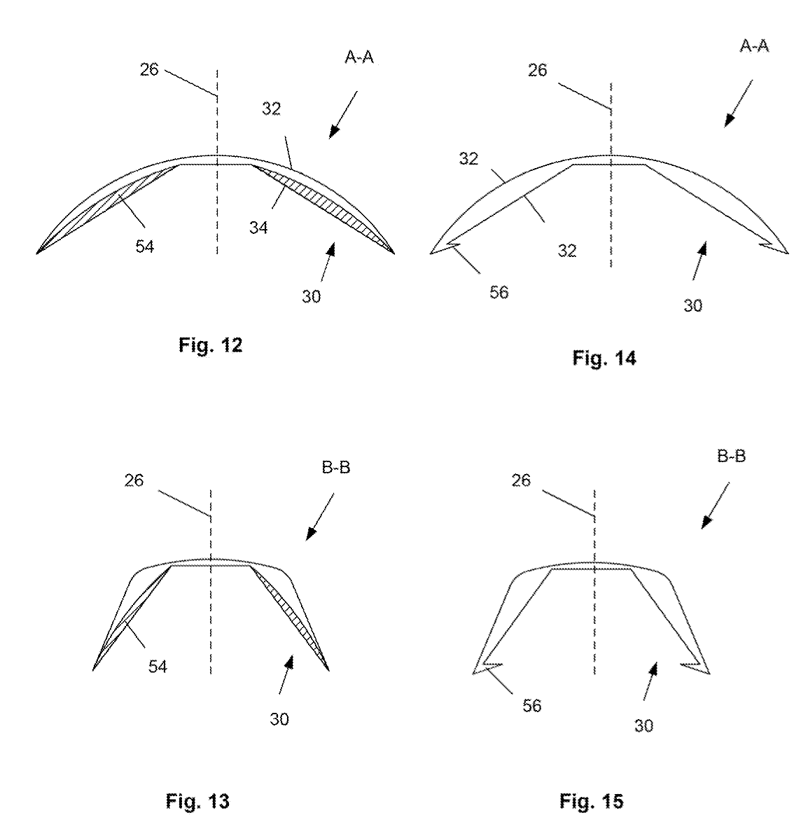

FIGS. 12 and 13 illustrate that the prosthesis can have an ingrowth matrix 54 integrated with or attached to the perimeter anchor 30. The ingrowth matrix 54 can form the proximal prosthesis surface 34. The central portion 28 can have or be absent of (as shown) ingrowth matrix 54.

FIGS. 14 and 15 illustrate that the distal prosthesis surface 32 can have an attacher. The attacher can be a ridge 56 (as shown), spine, prominence, rise, fin, skeg, or combinations thereof. The ridge 56 can be at the radial end away from the central axis 26. The ridge 56 can have a v-shaped configuration. The ridge 56 can be oriented radially inward toward the central axis 26. The ridge 56 can be configured to provide additional resistance in one direction (e.g., when pulling downward, as shown).

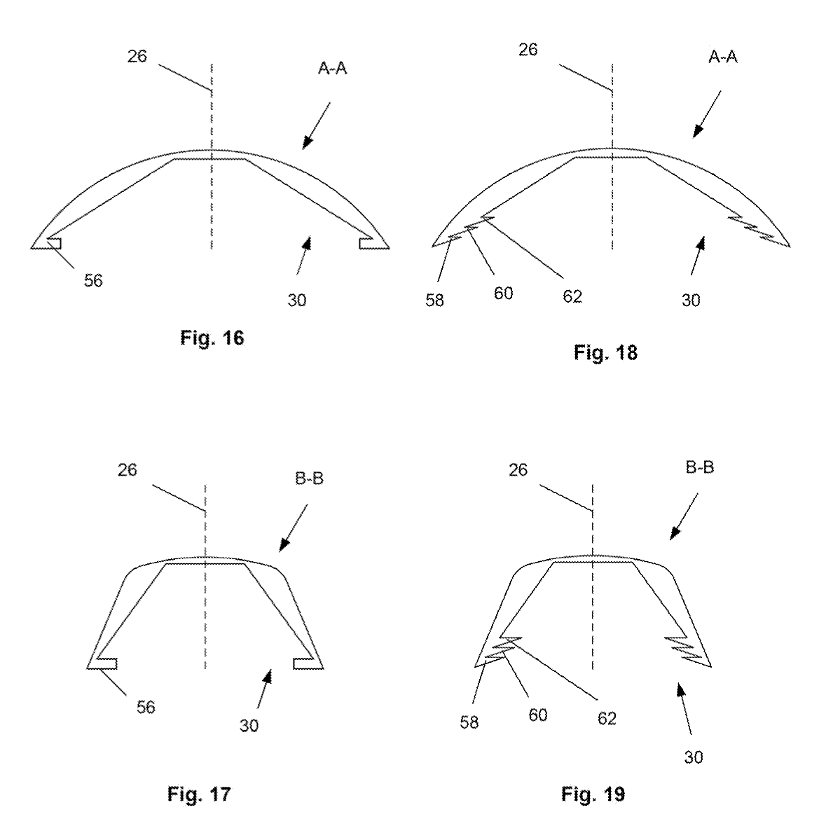

FIGS. 16 and 17 illustrate that the ridge 26 can have a t-shaped configuration. FIGS. 18 and 19 illustrate that the perimeter anchor can have first 58, second 60 and third ridges 62. The ridges 56 can be located progressively radially closer to the central axis 26.

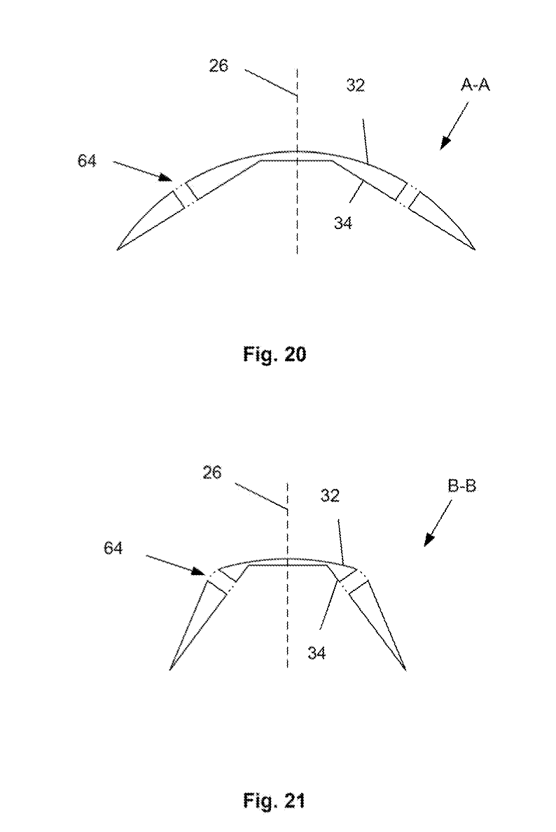

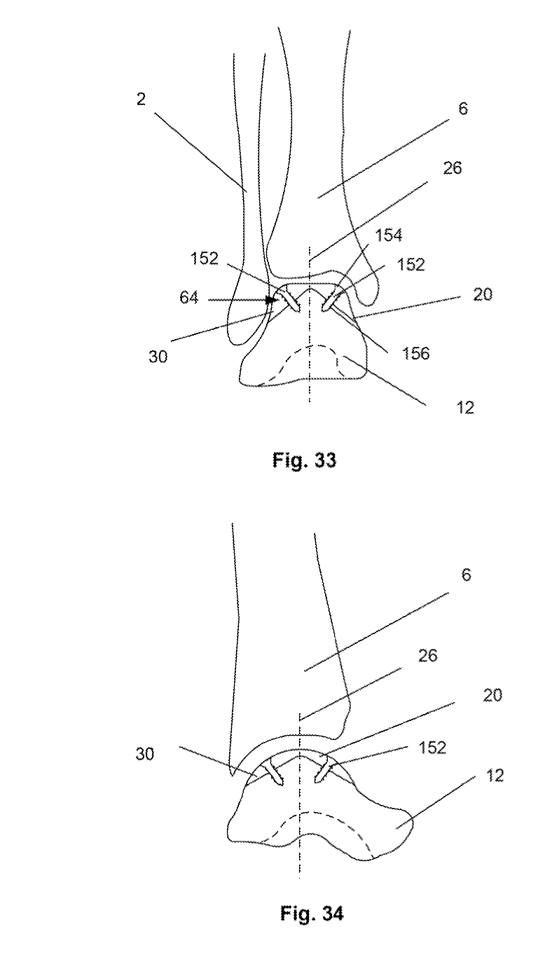

FIGS. 20 and 21 illustrate that the perimeter anchor 30 can have one or more (e.g., two or four) supplemental anchor ports 64. The supplemental anchor ports 64 can be between the distal prosthesis surface 32 and the proximal prosthesis surface 34. The supplemental anchor ports 64 can be threaded (shown in FIGS. 33 and 34). The supplemental anchor port 64 can be located at substantially the thickest portion of the perimeter anchor 30. The supplemental anchor port 64 can be configured perpendicularly to the proximal prosthesis surface 34.

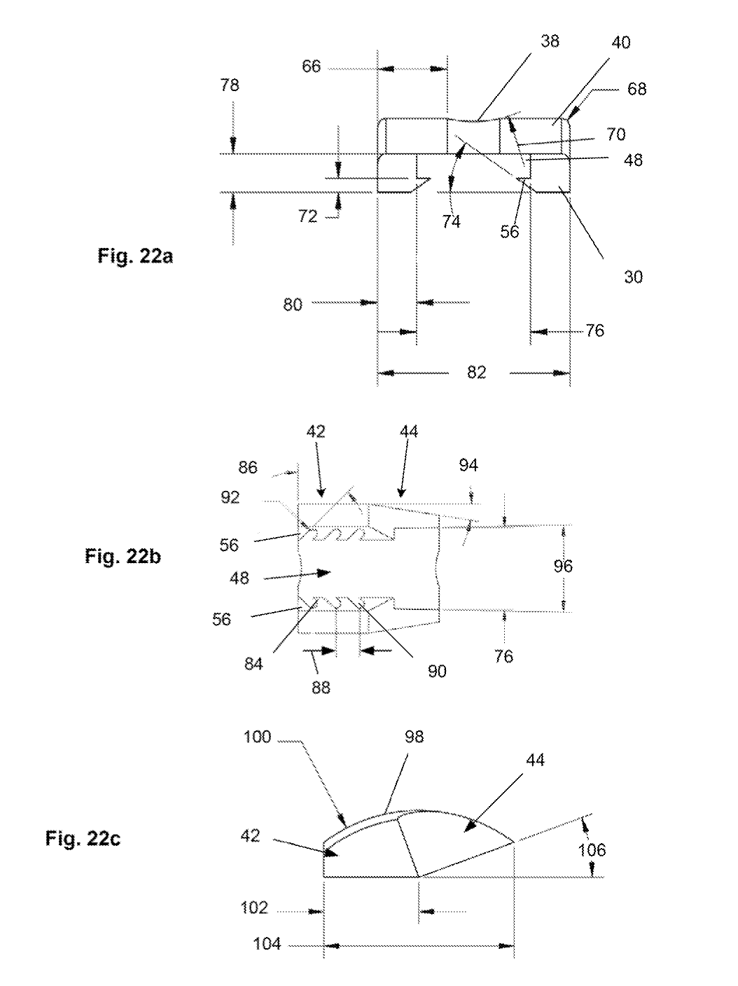

FIG. 22a illustrates that the shoulders 40 can have shoulder widths 66. The shoulder width 66 can be from about 6.4 mm (0.25 in.) to about 19 mm (0.75 in.), for example about 12.7 mm (0.500 in). The shoulders 40 can have a rounded transition to the sides of the prosthesis body 24 having a distal chamfer radios 68. The distal chamfer radius 68 can be from about 0.08 mm (0.03 in.) to about 3.0 mm (0.12 in.), for example about 2 mm (0.06 in.).

The groove 38 can have a groove radius 70 (of curvature). The groove radius 70 can be from about 10 mm (0.4 in.) to about 41 mm (1.6 in.), for example about 20.7 mm (0.813 in.).

The ridge 56 can have a ridge height 72 and a ridge angle 74. The ridge height 72 can be from about 1.3 mm (0.05 in.) to about 5 mm (0.2 in.), for example about 2.54 mm (0.100 in.). The ridge angle 74 can be from about 17° to about 70°, for example about 35°.

The bone channel 48 can have a bone channel width 76. The bone channel width 76 can be from about 10 mm (0.4 in.) to about 41 mm (1.6 in.), for example about 20.7 mm (0.813 in.).

The perimeter anchor 30 can have a perimeter anchor height 78 and a perimeter anchor width 80. The perimeter anchor height 78 can be from about 3.3 mm (0.13 in.) to about 14 mm (0.55 in.), for example about 6.99 mm (0.275 in.). The perimeter anchor width 80 can be from about 3.6 mm (0.14 in.) to about 14 mm (0.56 in.), for example about 7.14 mm (0.281 in.).

The prosthesis body 24 can have a prosthesis body width 82 from about 17 mm (0.68 in.) to about 69.9 mm (2.75 in.), for example about 34.9 mm (1.375 in.), also for example about 38 mm (1.5 in.).

FIG. 22b illustrates that the ridge 56 can have one, two, three, four or more teeth 84. The teeth 84 can be sharpened. The teeth 84 can have a tooth angle 86 with respect to the face of closer end of the prosthesis body 24. The tooth angle 86 can be from about 20° to about 80°, for example about 45°. The teeth 84 can be separated from each other by a tooth gap 88. The tooth gap 88 can be from about 2 mm (0.08 in.) to about 12 mm (0.5 in.), for example about 3.96 mm (0.156 in.), also for example about 6.35 mm (0.250 in.). The teeth 84 can have a tooth slot 90 between the teeth 84. The tooth slot 90 can have a tooth slot diameter 92 from about 12 mm (0.5 in.) to about 53 mm (2.1 in.), for example about 28 mm (1.1 in.).

The sides of the prosthesis rise 44 can taper at a rise taper angle 94 inward as it approaches the end of the prosthesis body 24. The rise taper angle 94 can be from about 0° to about 45°, more narrowly from about 4° to about 20°, for example about 9°.

The bone channel 48 can taper at a bone channel angle 96. The bone channel angle 96 can be from about 0° to about 10°, for example about 2.4°.

FIG. 22c illustrates that the distal surface 98 can have a distal surface radius 100 (of curvature). The distal surface radius 100 can be from about 15 mm (0.6 in.) to about 64 mm (2.5 in.), for example about 31.50 mm (1.240 in.).

The prosthesis flat 42 can have a prosthesis flat length 102. The prosthesis flat length 102 can be from about 8 mm (0.3 in.) to about 80 mm (3 in.), for example about 19.1 mm (0.750 in.). The prosthesis body 24 can have a prosthesis body length 104 from about 19 mm (0.75 in.) to about 80 mm (3 in.), for example about 38.10 mm (1.500 in.). The length of the prosthesis rise 44 can be the difference between the prosthesis fiat length 102 and the prosthesis body length 104: about 0 mm (0 in.) to about 69 mm (2.7 in.), for example about 38 mm (1.5 in.).

The prosthesis rise 44 can have a rise lift angle 106 with respect to the bottom of the prosthesis flat 42. The rise lift angle 106 can be from about 0° to about 45% more narrowly from about 10° to about 40°, for example about 20.2°.

FIG. 23 illustrates that the prosthesis floating component 108 can have a substantially square or rectangular transverse section. The prosthesis floating section 108 can have a tibia-side surface 110 opposite of a talus-side surface 112. A tibia tongue 114 can extend from the tibia-side surface 110. The tibia tongue 114 can be configured to act as a slidable guide within the groove on the tibia prosthesis. A talus tongue 116 can extend from the talus-side surface 112. The talus tongue 116 can be configured to act as a slidable guide within the groove on the talus prosthesis.

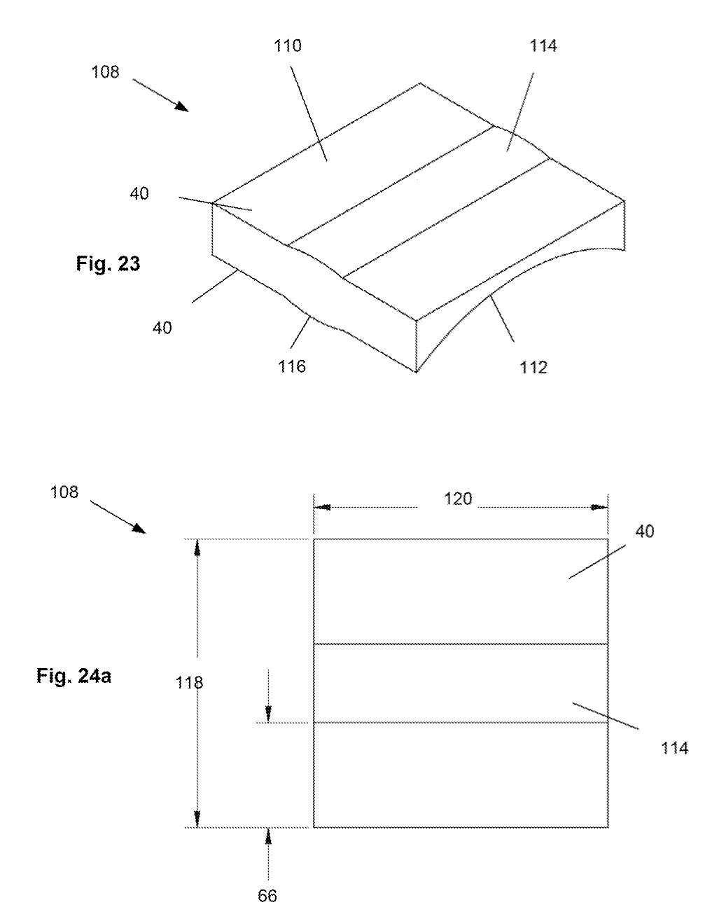

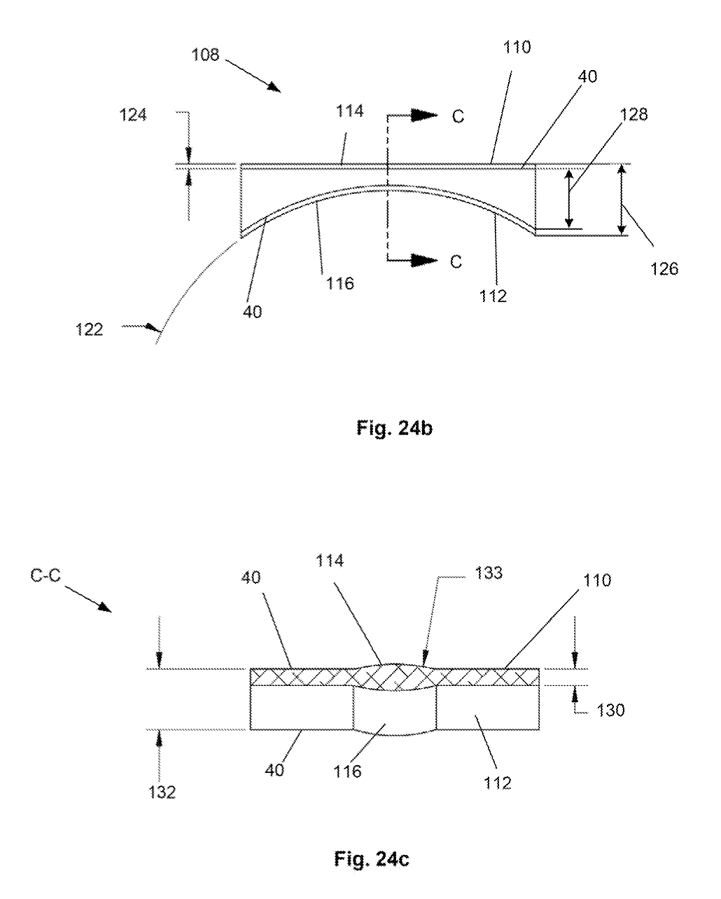

FIG. 24a illustrates that the prosthesis floating component 108 can have a floating component width 118 and a floating component length 120. The floating component width 118 can be from about 18 mm (0.7 in.) to about 71 mm (2.8 in.), for example about 34.93 in. (1.375 in.). The floating component length 120 can be from about 18 mm (0.7 in.) to about 71 mm (2.8 in.), for example about 36 mm (1.4 in.).

The one or more shoulders 40 on the prosthesis floating component 108 can each have a shoulder width 66 from about 6.4 mm (0.25 in.) to about 25 mm (1.0 in.). The tibia 114 and talus tongues 116 can have the about same widths as the corresponding grooves 38 in the respective prosthesis components.

FIG. 24b illustrates that the shoulders 40 on the tibia-side surface 110 can be substantially flat. The talus tongue 116 and the shoulders 40 on the talus-side surface 112 can have a talus-side radius 122 (of curvature). The talus-side radius 122 can be from about 15 mm (0.6 in.) to about 64 mm (2.5 in.), for example about 32.13 mm (1.265 in.).

The talus-side surface 112 can be flat. The tibia-side surface 110 can be rounded.

The tongues 114, 116 can have a tongue height 124. The tongue height 124 can be from about 0.3 mm (0.01 in.) to about 1.3 mm (0.05 in.), for example about 5.6 mm (0.022 in.).

The floating component 108 can have a floating component height 126. The floating component height 126 without the tongues 114, 116 can be a tongueless height 128. The floating component height 126 can be from about 1.5 mm (0.06 in.) to about 17 mm (0.68 in.), for example about 8.43 mm (0.332 in.).

FIG. 24c illustrates that the prosthesis can have a minimum tongueless height 130, and a maximum tongueless height 132. The minimum tongueless height 139 can be about at the mid-point from front to back of the prosthesis Boating component 108. The minimum tongueless height 130 can be from about 1 mm (0.04 in.) to about 4.1 mm (0.16 in.), for example about 2.0 mm (0.079 in.). The maximum tongueless height 132 can be about at the front and/or back ends of the prosthesis floating component 108. The maximum tongueless height 1.32 can be from about 3.6 mm (0.14 in.) to about 15 mm (0.58 in.), for example about 7.32 mm (0.288 in.).

The tongues 114, 116 can have the same or different tongue radii 133. The tongue radii 133 can be from about 10 mm (0.4 in.) to about 41 mm (1.6 in.), for example about 20.7 mm (0.813 in.). The tongue radii 133 can be about equal to the groove radii on the adjacent prosthesis component. For example, the groove radius 70 for the prosthesis tibia component 134 can be about the same as the tongue radius 133 for the tongue tibia-side surface 110 of the prosthesis floating component 108. The groove radius 70 for the prosthesis talus component can be about the same as the tongue radius 133 for the tongue talus-side surface 110 of the prosthesis floating component 108.

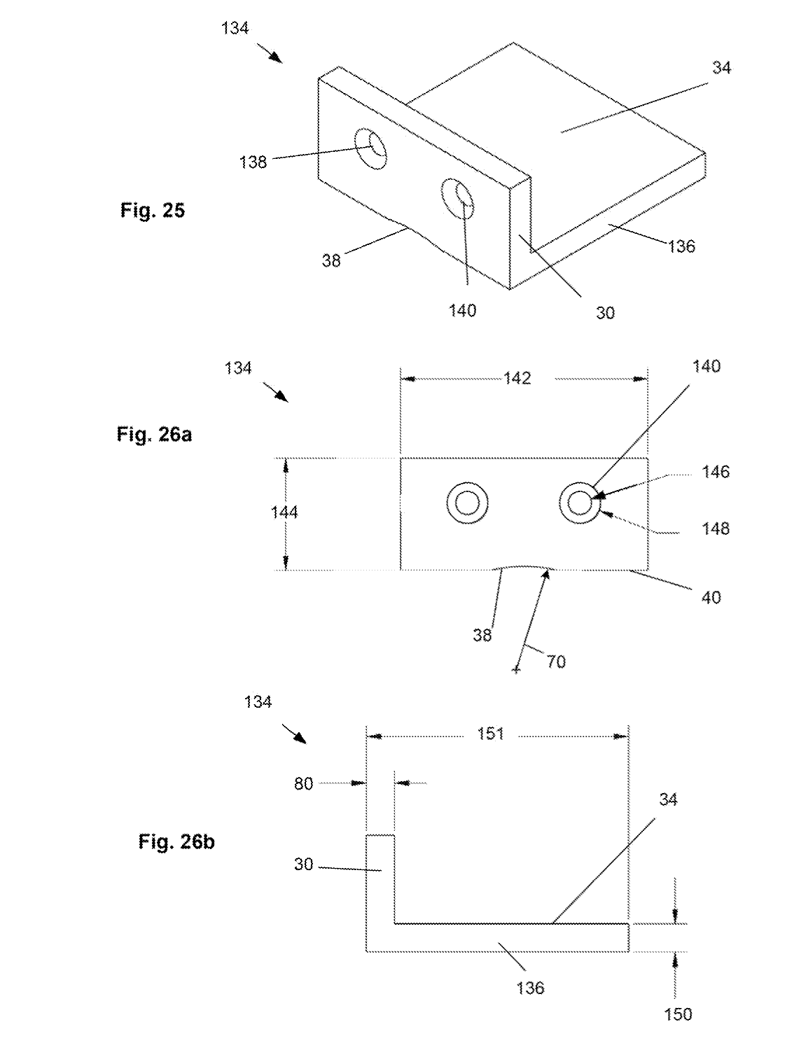

FIG. 25 illustrates a prosthesis tibia component 134 that can have a perimeter anchor 30 that extends from a base 136 along a single side of the base 136. The perimeter anchor 30 can extend at a right, obtuse, or acute angle from the base 136. The perimeter anchor 30 can extend from one, two, three, four or more sides of the base 136. The perimeter anchor 30 can have a first supplemental anchor port 138 and a second supplemental anchor port 140. The anchor ports 138, 140 can be straight or tapered. The anchor ports 138, 140 can be threaded or unthreaded. The prosthesis tibia component 134 can have a groove 38 configured to slidably engage the tibia tongue 114 on the prosthesis floating component 108. The prosthesis tibia component 134 can have a tongue 114, 116 configured to slidably engage the groove 38 on the prosthesis talus component 158, for example when in use without the prosthesis floating component 108.

The tongues 114, 116 on the prosthesis floating component 108 can either or both be grooves 38, and the grooves 38 on the prosthesis tibia component 134 and the prosthesis talus component 158 can either or both be tongues 114, 116 to engagably match the corresponding structure on the prosthesis floating component 108.

FIG. 26a illustrates that the prosthesis tibia component 134 can have a tibia component length 142 and a tibia component height 144. The tibia component length 142 can be from about 17 mm (0.65 in.) to about 69 mm (2.7 in.), for example about 34.93 mm (1.375 in.). The tibia component height 144 can be from about 7.9 mm (0.31 in.) to about 31.8 mm (1.25 in.), for example about 15.9 mm (0.625 in.).

The anchor ports 138, 140 can have an anchor port inner radius 146 and an anchor port outer radius 148, for example if the anchor port is tapered or threaded. The anchor port inner radius 146 can be from about 1.5 mm (0.06 in.) to about 3.3 mm (0.13 in.), for example about 1.7 mm (0.065 in.). The anchor port outer radius 148 can be from about 1.5 mm (0.06 in.) to about 5.8 mm (0.23 in.), for example about 2.87 mm (0.113 in.).

The groove radius 70 of the prosthesis tibia component 134 can be about equal to the groove radius 70 for the prosthesis talus component 158.

FIG. 26b illustrates that the perimeter anchor 30 can have a perimeter anchor width 80. The perimeter anchor width 80 can be from about 2 mm (0.07 in.) to about 8 mm (0.3 in.), for example about 3.81 mm (0.150 in.).

The base 136 can have a base height 150. The base height 150 can be from about 2 mm (0.07 in.) to about 8 mm (0.3 in.), for example about 3.81 mm (0.150 in.).

The prosthesis tibia component 134 can have a tibia component width 151. The tibia component width 151 can be from about 17 mm (0.7 in.) to about 71 mm (2.8 in.), for example about 35.56 mm (1.400 in.).

Any or all elements of the prosthesis and/or other devices or apparatuses described herein, including the prosthesis body 24 of the talus prosthesis, prosthesis floating component 108, and/or tibial prosthesis, or any other prosthesis, can have a surface finish to about 1.6 μm (63 μin.) or less.

Any or all elements of the prosthesis and/or other devices or apparatuses described herein, including the prosthesis body 24 of the talus prosthesis, prosthesis floating component 108, and/or tibial prosthesis, or any other prosthesis, can be made from, for example, a single or multiple stainless steel alloys, nickel titanium alloys (e.g., Nitinol), other titanium alloys, cobalt-chrome alloys (e.g., ELGILOY® from Elgin Specialty Metals, Elgin, Ill.; CONICHROME® from Carpenter Metals Corp., Wyomissing, Pa.), aluminum and aluminum alloys (e.g., 6060-T6 aluminum, 6061-T6 aluminum), nickel-cobalt alloys (e.g., MP35N® from Magellan Industrial Trading Company, Inc., Westport, Conn.), molybdenum alloys (e.g., molybdenum. TZM alloy, for example as disclosed, in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET)/polyester (e.g., DACRON® from E. I. Du Pont de Nemours and Company, Wilmington, Del.), polypropylene, (PET), polytetrafluoroethylene (PTFE), expanded PTFE (ePTFE), polyether ether ketone (PEEK), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), polylactic acid (PLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, bone morphogenic protein (BMP), osteogenic powder, beads of bone) any of the other materials listed herein or combinations thereof Examples of radiopaque materials are barium sulfate, zinc oxide, titanium, stainless steel nickel-titanium alloys, tantalum and gold.

Any or all elements of the prosthesis and/or other devices or apparatuses described herein can be or have a matrix for cell ingrowth 54 (e.g., as described supra) or used with a fabric, for example a covering (not shown) that acts as a matrix for cell ingrowth 54. The matrix and/or fabric can be, for example, polyester (e.g., DACRON®) from E. I. Du Pont de Nemours and Company, Wilmington, Del.), polypropylene, PTFE, ePTFE, nylon, extruded collagen, a cobalt-chrome alloy matrix, silicone or combinations thereof.

The elements of the prosthesis and/or other devices or apparatuses described herein and/or the fabric can be filled and/or coated with an agent delivery matrix known to one having ordinary skill in the art and/or a therapeutic and/or diagnostic agent. The agents within these matrices can include radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Any or all parts of the prosthesis or other elements, tools, bones or other parts of the implant site can be coated with hydroxyapetite. Examples of other agents are provided in Walton et al, Inhibition of Prostoglandin E₂ Synthesis in Abdominal Aortic Aneurysms, Circulation, Jul. 6, 1999, 48-54; Tambiah et al. Provocation of Experimental Aortic Inflammation Mediators and Chlamydia Pneumoniae, Brit. J. Surgery 88 (7), 935-940; Franklin et al. Uptake of Tetracycline by Aortic Aneurysm Wall and Its Effect on Inflammation and Proteolysis, Brit. J. Surgery 86 (6), 771-775; Xu et al, Sp1 Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

### METHOD OF USE

The prosthesis 20 can be used at the end of long bones. The prosthesis 20 can be used on the superior side of the talus 12. The prosthesis 20 can be used on vertebra.

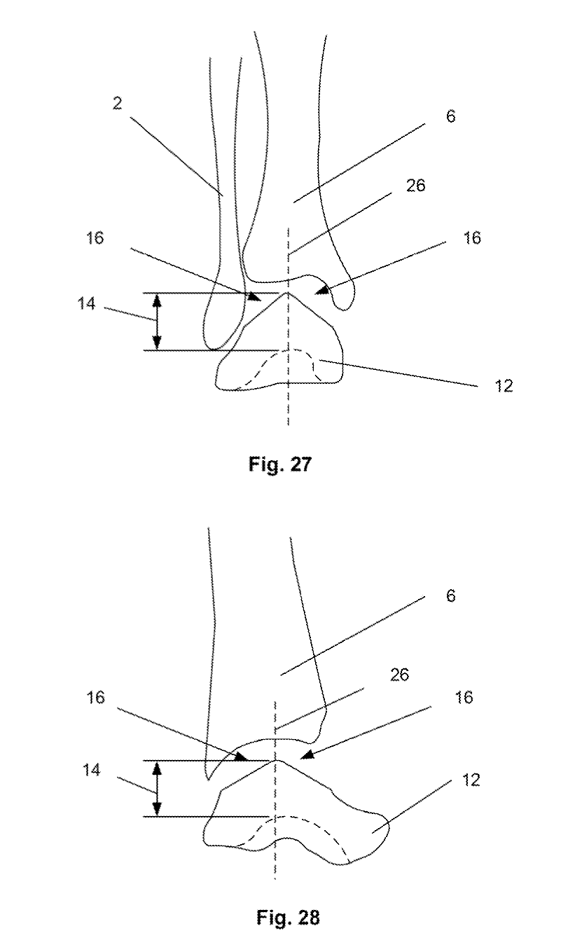

FIGS. 27 and 28 illustrate the superior surface of the talus 12—or bone end of a long bone (not shown)—can have tissue removed to prepare for the implantation of the prosthesis 20. More tissue can be removed from the superior surface of the bone radially distal from the central axis 26 than radially proximal to the central axis 26.

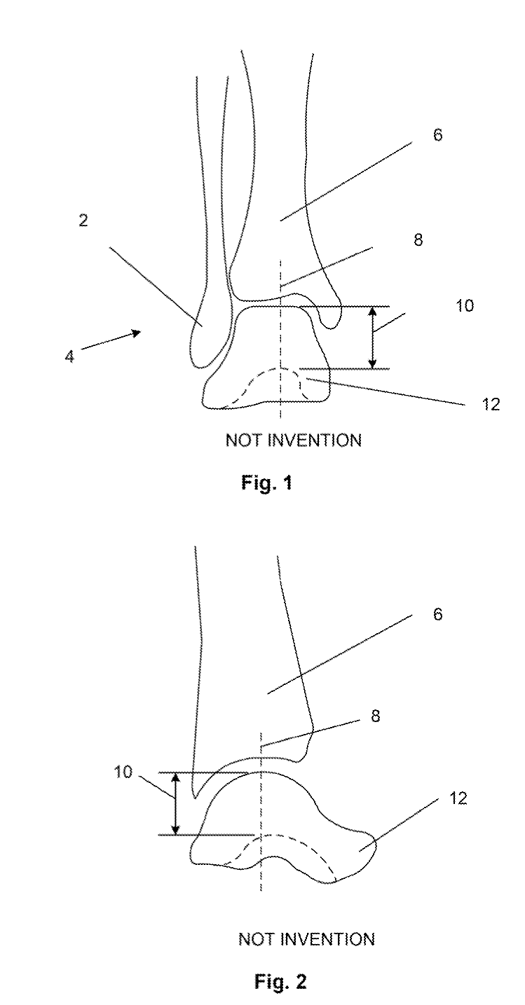

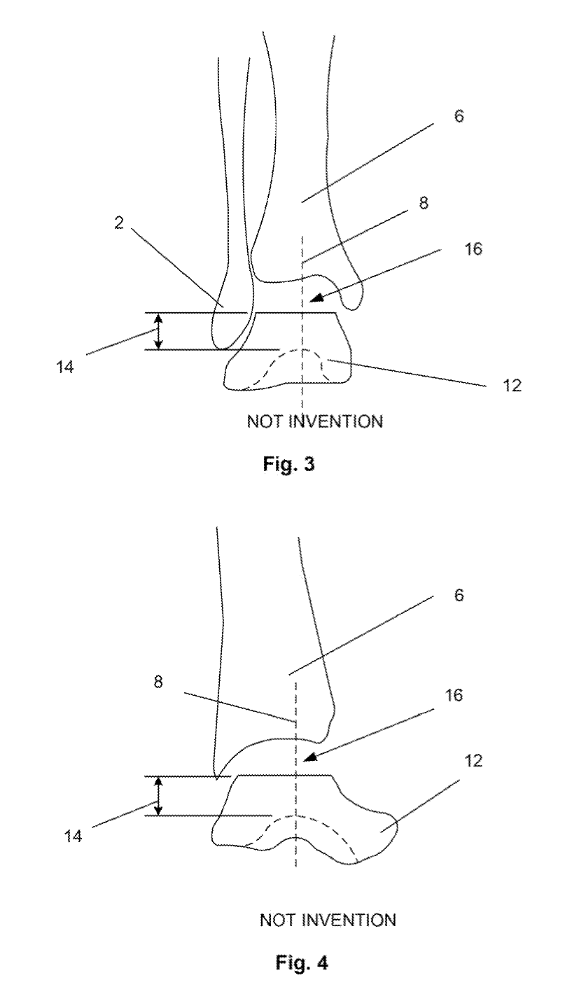

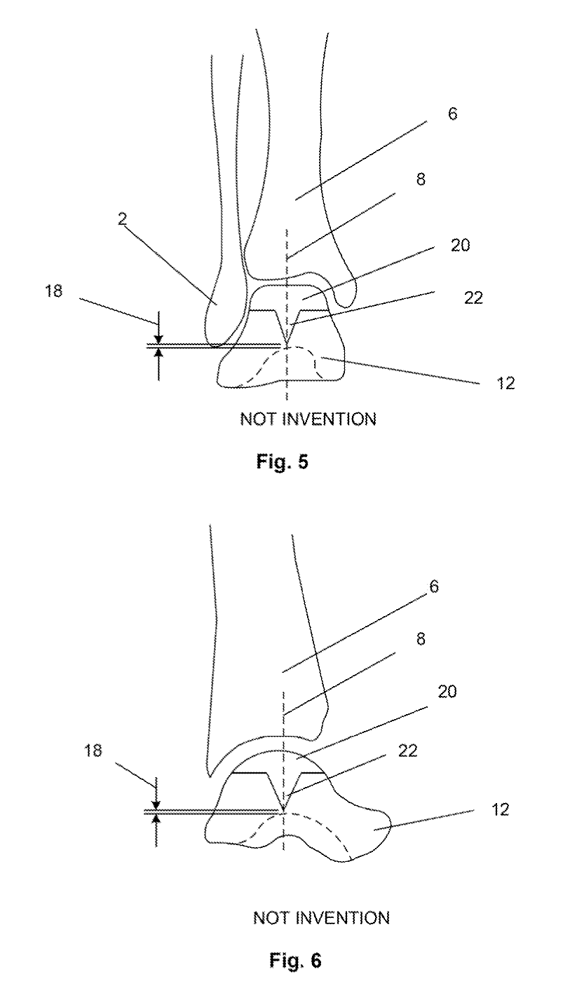

The talus 12 can have a minimum cut-down thickness 16. The minimum cut-down thickness 16 can be substantially equivalent to the original talus thickness 10 shown in FIGS. 1 and 2. The minimum cut-down thickness 16 can be substantially larger than the minimum cut-down thickness 16 shown in FIGS. 3 and 4.

All or part of the remaining superior surface can be a contact patch 16, for example, for the prosthesis 20 to attach to in whole or part. BMP known to those having ordinary skill in the art can be applied to the contact patch 16 and/or to the surface (e.g., the surface that will contact the contact patch 16) of the prosthesis 20, for example the perimeter anchor 30, before or after the prosthesis 20 is attached to the talus 12.

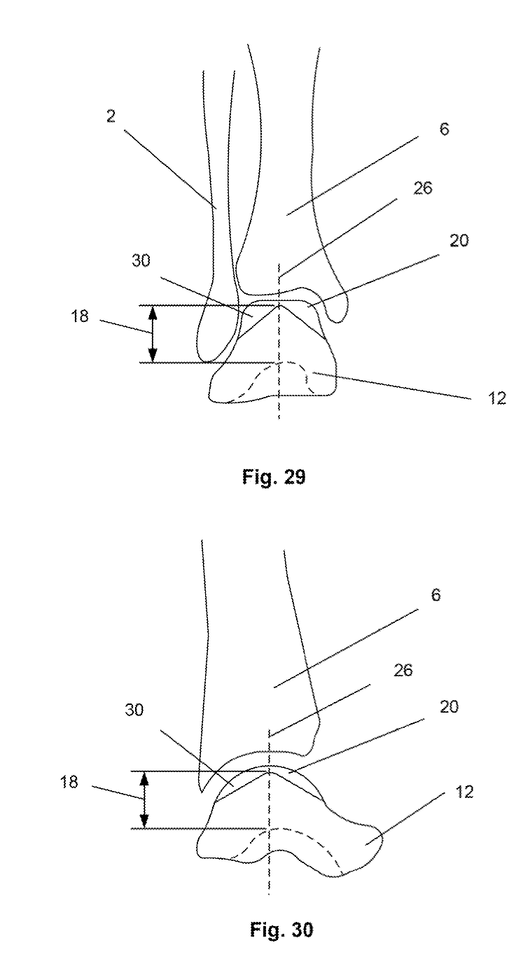

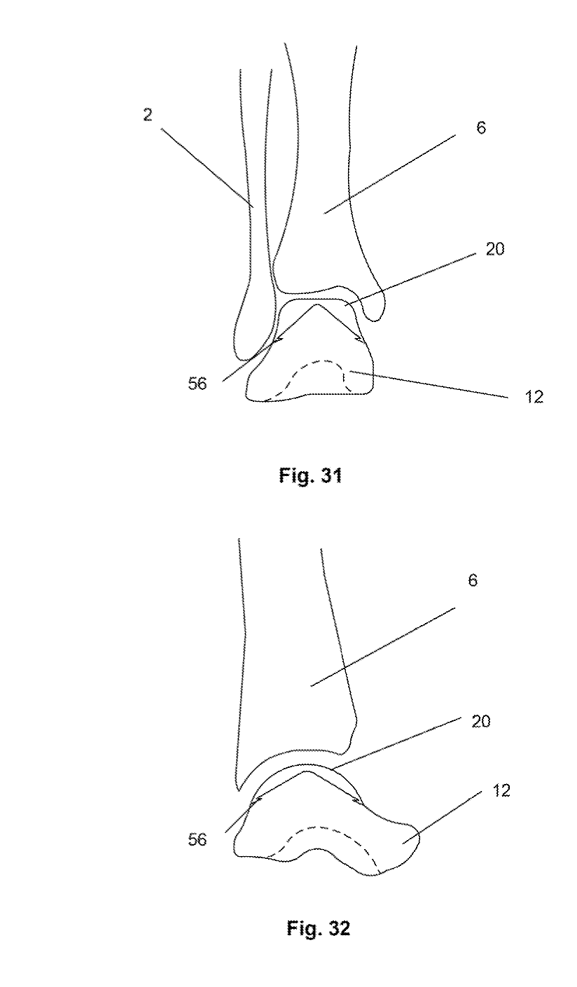

FIGS. 29 and 30 illustrate that the prosthesis 20 can be attached to ail or part of the superior surface of the talus 12. The perimeter anchor 30 can attach to the talus 12. A post-implant talus minimum thickness 18 can be substantially equivalent to the minimum cut-down thickness 14 of FIGS. 27 and 28.

FIGS. 31 and 32 illustrates that the ridge 56 can attach to the talus 12. The talus 12 can have tissue removed in a configuration to mate the ridge 56. The talus 12 can have no tissue removed to mate the ridge 56, for example, and the ridge 56 can deform the talus 12 and/or the prosthesis 20.

FIGS. 33 and 34 illustrate that one or more supplemental anchors 152 can be deployed through the prosthesis 20 and into or through the talus 12. The supplemental anchor 152 can be deployed through the perimeter anchor 30 and/or through the central portion 28. The supplemental anchors 152 can be screws, pins, brads, sutures, or combinations thereof.

The supplemental anchor 152 can have a supplement anchor head 154. The end of the supplemental anchor port 64 adjacent to the distal prosthesis surface 32 can be sunken to receive the supplemental anchor head 154 in a configuration flush with the distal prosthesis surface 32.

The supplemental anchor 152 can have supplemental anchor thread 154. The supplemental anchor thread 154 can be received by a thread in the supplemental anchor port 64. The supplemental anchor thread 154 can be configured to fixedly or removably attach to the talus 12.

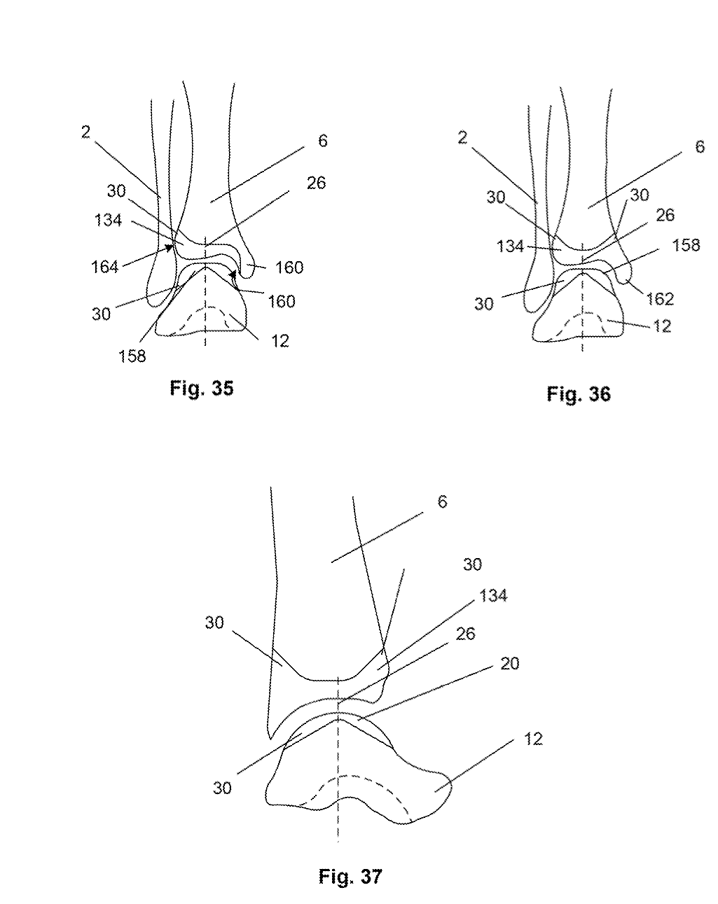

FIGS. 35 through 36 illustrate a method of Implanting the prosthesis 20 that can have multiple components. The prosthesis 20 can have a prosthesis talus component 158 and a prosthesis tibia component. The prosthesis talus component 158 can, for example, be substantially equivalent to any of the prostheses 20 shown in FIGS. 7-22c and 29-34. The prosthesis tibia component 134 can have the perimeter anchor 30. The perimeter anchor 30 of the prosthesis tibia component 134 can ascend superiorly along the lateral, anterior and posterior perimeter of the tibia 6, as shown in FIGS. 35 through 37. The perimeter anchor 30 of the prosthesis tibia component 134 can descend interiorly and/or terminate along the medial malleolus articular facet 160, as shown in FIG. 35. The perimeter anchor 30 of the prosthesis tibia component 134 can ascend superiorly along the medial perimeter of the tibia 6, as shown in FIG. 36. The perimeter anchor 30 of the prosthesis tibia component 134 can replace the medial malleolus 162, as shown In FIG. 36.

The medial malleolus 162 can be completely or partially preserved or completely or partially removed. For example, the prosthesis tibia component 134 can bolster the medial malleolus 162, as shown in FIG. 35. The prosthesis tibia component 134 can replace the medial malleolus 162, as shown in FIG. 36. The prosthesis tibia component 134 can be preserved, for example, with the use of a properly sized prosthesis tibia component 134 as shown in FIG. 25.

The prosthesis tibia component 134 can be configured to approximate the fibular notch 164, as shown in FIGS. 35 and 36.

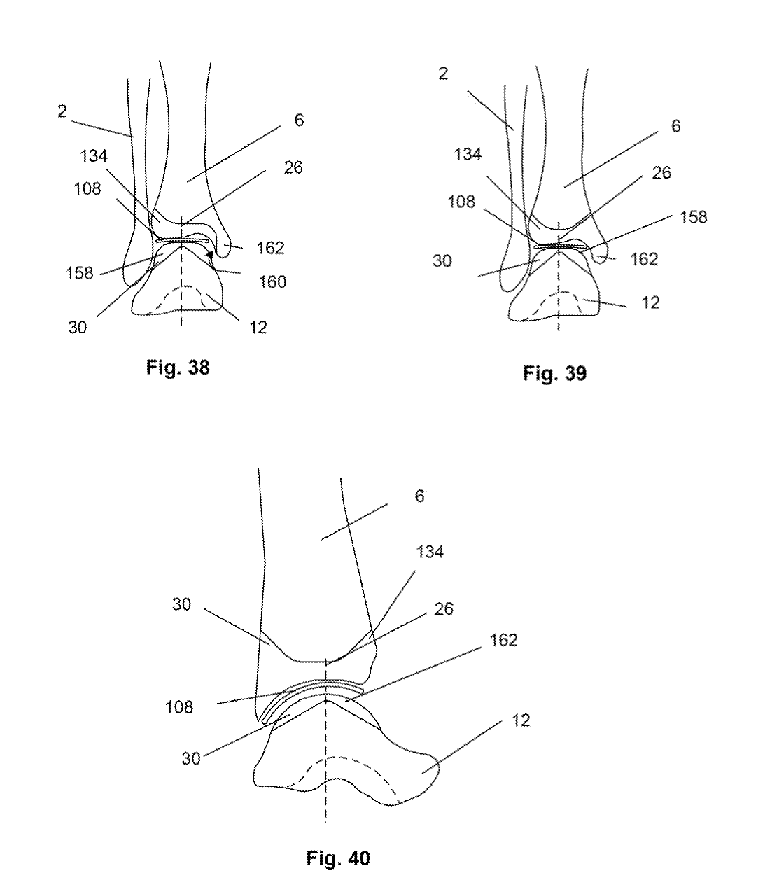

FIGS. 38 through 40 illustrate that the prostheses 20 can have a prosthesis floating component 108. The prosthesis floating component 108 can be separate from the prosthesis talus component 158 and the prosthesis tibia component 134. The prosthesis floating component 108 can be implanted between the prosthesis talus component 158 and the prosthesis tibia component 134. The prosthesis floating component 108 can be configured to slidably contact the prosthesis talus component 158 and the prosthesis tibia component 134. The prosthesis floating component 108, the prosthesis tibia component 134, and the prosthesis talus component 158 can be made from the same and/or different materials.

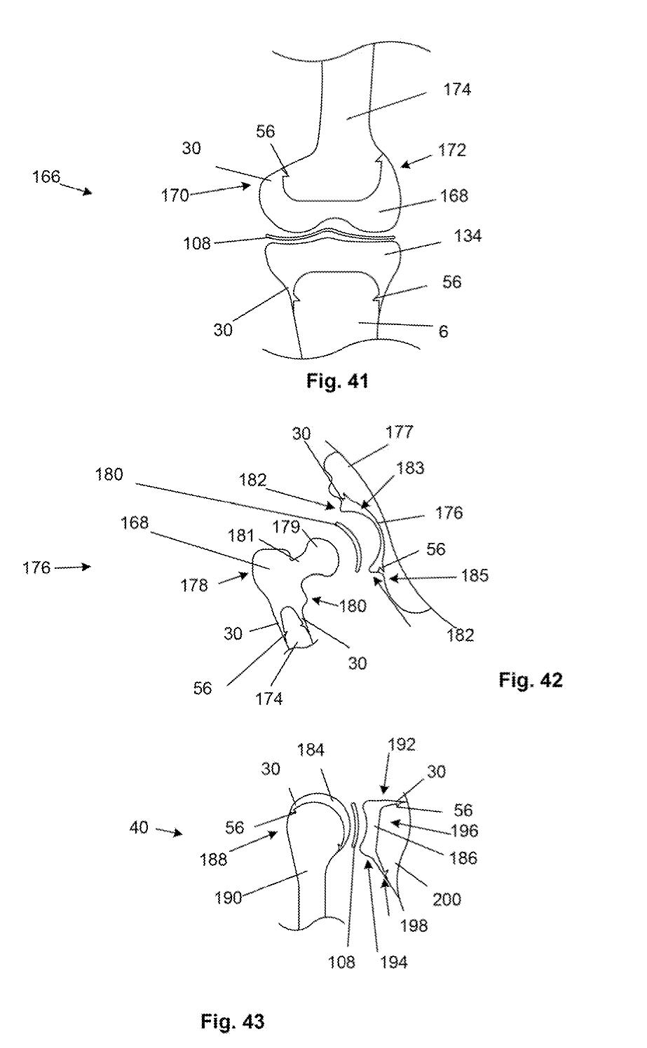

FIG. 41 illustrates that the prosthesis 20 can be implanted in a knee 166. The prosthesis 20 can have a prosthesis femur component 168, a prosthesis tibia component 134, a prosthesis floating component 108, or combinations thereof. The prosthesis femur component 168 can be configured to replace, and extend superiorly to and/or beyond, the adductor tubercle 170 and lateral epicondyle 172 around part of or substantially or completely the entire perimeter of the femur 174. The prosthesis tibia component 134 can be configured to replace, and extend inferiorly beyond, the Gerdy's tubercle around part of or substantially or completely the entire perimeter of the tibia 12.

FIG. 42 illustrates that the prosthesis 20 can be implanted in a hip 176, notably in the femur and pelvis 177. The prosthesis 20 can have a prosthesis femur component 168, a prosthesis pelvis component 176, a prosthesis floating component 108, or combinations thereof. The prosthesis femur component 168 can be configured to replace, and extend inferiorly to and/or beyond, the greater trochanter 178 and lesser trochanter 180 around part of or substantially or completely the entire perimeter of the femur 174. The prosthesis femur component 168 can be configured to approximate or otherwise mimic the anatomical features including the greater trochanter 178, lesser trochanter 180, femur head 179 and femur neck 181.

The prosthesis pelvis component 176 can be configured to replace, and extend radially beyond, the acetabular notch 182. The prosthesis pelvis component 176 can be configured to approximate or otherwise mimic or transition smoothly into the anatomic features including the acetabulum 183, and superior pubis ramus 185.

FIG. 43 illustrates that the prosthesis 20 can be implanted in a shoulder 40. The prosthesis 20 can have a prosthesis humerus component 184, a prosthesis scapula component 186, a prosthesis floating component 108, or combinations thereof. The prosthesis humerus component 184 can be configured to replace, and extend superiorly to and/or beyond, the greater tubercle 188 and/or lesser tubercle around part of or substantially or completely the entire perimeter of the humerus 190. The prosthesis scapula component 186 can be configured to replace, and extend inferiority beyond, the supraglenoid tubercle 192, infraglenoid tubercle 194, the neck of the scapula 196, and/or the triceps brachii attachment 198 around part of or substantially or completely the entire perimeter of the scapula 200.

During use of the implant in weight hearing joints (e.g., ankle, knee, hip, vertebrae), the patient can walk to force the prosthesis against the bone to which the prosthesis is implanted.

The prosthesis can be sized so that during preparation (e.g., as shown in FIGS. 27 and 28) of the talus and implantation and attachment of the prosthesis that the removal or other loss of existing talus be minimal, for example less than about 35% of the talus volume, more narrowly less than about 20% of talus volume, yet more narrowly less than 10% of the talus volume. The prosthesis can be sized to cause no or minimal damage to the cancellous bone, for example minimizing or preventing impairment of blood flow to, from and within the bone and blood cell generation by the bone.

The prosthesis can be attached to the talus, without creating a hole substantially completely through the talus with substantially opposite ends of the hole in the direction of the dorsal-plantar axis of the talus and/or completely surrounded by the bone except for the exit and entry port of the hole.

It is apparent to one skilled in the art that various changes and modifications can be made to this disclosure, and equivalents employed, without departing from the spirit and scope of the invention. Elements shown with any variation are exemplary for the specific variation and can be used in combination with, or otherwise on or in, other variations within this disclosure. An example of combination would be a prosthesis that can have the ridges of the prosthesis shown in FIGS. 18 and 19 and the ingrowth matrix shown of the prosthesis shown in FIGS. 12 and 13. The ingrowth matrix can extend down the prosthesis to some, all, or none of the ridges.

Any or all surfaces or portions of surfaces of the prosthesis can be configured to allow ingrowth and/or supplemental anchoring of soft tissue (e.g., ligament, tendon, membrane).

Anatomical representations in the figures have been simplified for the purpose of illustration. One having ordinary skill in the art can identify the anatomical details implied but not explicitly shown and extrapolate and interpolate therefrom to understand and enable the disclosure herein. For example, though the trochlea, various facets and asymmetry are not shown, one having ordinary skill in the art understands that the prosthesis fits and mimics the configurations of the trochlea, various facets and asymmetry where appropriate.

1. A partial bone prosthesis device comprising:
a prosthesis body having a central axis, a central portion and a perimeter anchor, wherein the central axis passes through the central portion, and wherein the perimeter anchor is radially distal to the central axis.

2. The device of claim 1, wherein the perimeter anchor is substantially thicker than the central portion.

3. The device of claim 1, wherein the perimeter anchor forms a complete perimeter around the central portion.

4. The device of claim 1, wherein the perimeter anchor forms an incomplete perimeter around the central portion.

5. The device of claim 4, wherein the prosthesis body comprises a branch.

6. The device of claim 5, wherein the branch comprises the perimeter anchor.

7. The device of claim 1, wherein the perimeter anchor comprises an ingrowth matrix.

8. The device of claim 1, wherein the perimeter anchor comprises a first ridge.

9. The device of claim 8, wherein the perimeter anchor comprises a second ridge.

10. The device of claim 1, wherein the perimeter anchor comprises a supplemental anchor port.

11. The device of claim 1, wherein the pros thesis body comprises titanium

12. The device of claim 1, wherein the prosthesis body comprises cobalt chromium.

13. The device of claim 1, wherein the prosthesis body comprises bone morphogenic protein.

14. A method for implanting a partial bone prosthesis to a bone end of a bone having a central axis, comprising:
removing tissue from the bone end, wherein removing tissue comprises removing substantially thicker tissue distally from the central axis than proximally to the central axis, and wherein removing tissue comprises exposing a contact patch; and
deploying the partial bone prosthesis to the contact patch.

15. The method of claim 14, wherein deploying comprises applying bone morphogenic protein.

16. The method of claim 15, wherein deploying comprises inserting a supplemental anchor through the partial bone prosthesis and into the bone.

17. The method of claim 16, wherein inserting comprises inserting the supplemental anchor through the anchor perimeter.

18. A method for implanting a partial bone prosthesis to a talus, comprising:
attaching the prosthesis to the talus, wherein the attaching does not include creating a hole substantially completely through the talus in the direction of the dorsal-plantar axis of the talus.

19. The method of claim 18, wherein the hole is completely surrounded by the bone except for two substantially opposite ends of the hole.

20. A method for implanting a partial bone prosthesis to a talus, comprising:
removing a portion of the talus, and
attaching the prosthesis to the talus,
wherein the amount of talus removed by the removal of the portion of the talus and the attaching the prosthesis to the talus is less than 35% of the talus.

21. The method of claim 20, wherein the amount of talus removed by the removal of the portion of the talus and the attaching the prosthesis to the talus is less than 20% of the talus.
